(19) United States
(12) Patent Application Publication
(10) Pub. No.: US 2026/0188439 A1
(43) Pub. Date: Jul. 2, 2026

(54) AUXILIARY SCORING SYSTEM WITH SCORING CONSENSUS AND METHOD THEREOF

(71) Applicant: INDUSTRIAL TECHNOLOGY RESEARCH INSTITUTE, Hsinchu (TW)

(72) Inventors: Yu-Lien Shen, Taichung City (TW); Jian-Ren Chen, Hsinchu City (TW); Yue-Min Jiang, Hsinchu City (TW)

(73) Assignee: INDUSTRIAL TECHNOLOGY RESEARCH INSTITUTE, Hsinchu (TW)

(21) Appl. No.: 19/004,005

(22) Filed: Dec. 27, 2024

(51) Int. Cl. G16H 10/20; G06N 3/09

(52) U.S. Cl. CPC G16H 10/20; G06N 3/09

(57) ABSTRACT

An auxiliary scoring method with scoring consensus, including: providing a plurality of pieces of answer data and a plurality of pieces of scoring data corresponding to the plurality of answer data as an input dataset; inputting the input dataset into a neural network model to train the neural network model; validating the neural network model, and assessing the accuracy of an output prediction of the neural network model to establish an artificial intelligence model; generating a plurality of attention maps and/or a plurality of feature maps by using the artificial intelligence model; obtaining scoring consensus information based on the plurality of attention maps and/or the plurality of feature maps; and visualizing the scoring consensus information, and marking consensus blocks and non-consensus blocks of the scoring consensus information in different colors.

### TECHNICAL FIELD

The present invention relates to an artificial intelligence auxiliary scoring system and method, and in particular to, an artificial intelligence auxiliary scoring system and method with scoring consensus.

### BACKGROUND

Objective structured clinical examination (OSCE) is a critical link in cultivating professional medical talents. However, OSCE involves investment of significant resources, including time, manpower, money, and facilities. Therefore, its operating costs are extremely high. OSCE is intended to comprehensively assess clinical skills of students by simulating clinical contexts. Every year, a large number of medical students in various medical fields such as dentistry, internal medicine and surgery need to take this examination. OSCE typically involves that multiple scorers (e.g., examiners or teachers) score the performance of testees (e.g., examinees or students), including an objective part based on clear criteria, as well as some subjective judgment links. Since the examiners have different professional backgrounds, experience, personal preference or understanding of criteria, these factors may lead to different scoring results. For example, in the assessment of dental operating skills, the evaluation of the examiners on subjective criteria such as aesthetics and surface treatment details will often vary from person to person. The inconsistency of scoring criteria will affect the final score of the testee and will also affect his/her learning effect, which is not only unfavorable to individual testees, but also cannot achieve the evaluation goal of cultivating medical professionals. Especially in a case of limited resources, the limited teaching resources can not be effectively utilized, which also undermines the fairness and representativeness of the examination and leads to the waste of resources.

In addition, OSCE of each discipline has its own scoring principles, including objective and subjective judgment items. Taking the scoring principles of dental OSCE as an example, they can be roughly divided into objective formula-based judgment, objective formula-free judgment, subjective principled judgment and subjective unprincipled judgment. The scoring criteria of such scoring content often vary depending on differences in personal experience, professional backgrounds, and degrees of familiarity with specific clinical skills among the scorers. This scoring method based on the free evaluation of evidence of the scorers has a significant impact on the fairness of the examination. Therefore, in the OSCE link, the scorers often need to attend a consensus conference to discuss the uniformity of the scoring criteria, to ensure that different examiners have a consistent scoring criterion for the same skill or performance, thereby reducing the difference in subjective scoring. In addition, the process of the consensus conference is often very time-consuming and labor-intensive. On the one hand, OSCE scoring is highly complex. Therefore, in order to reach a consensus among the scorers, multiple discussions are often required, which involves both repeated amendments of the scoring criteria and in-depth discussions of the details of each skill. On the other hand, the time cost of the consensus conference is high, especially in the context of large-scale examinations, and it takes quite a long time to coordinate and adjust opinions from multiple scorers. With the widespread use of OSCE in medical education, the importance of the consensus conference for the scorers is increasing day by day, but due to the different professional backgrounds and teaching experience of each scorer, quite a long time and effort are often required for consistent scoring criteria. Moreover, such a consensus conference will be extended for disputes over the details, so that the balance between scoring consistency and efficiency becomes more difficult.

### SUMMARY

In view of this, the present invention provides an auxiliary scoring system and method with scoring consensus. An artificial intelligence model is utilized to analyze consensus or non-consensus (or differences) of scorers, and scoring consensus information is presented in a visualized manner, so as to assist discussions in a consensus conference, thereby accelerating to find out common scoring criteria and rules among the scorers. Therefore, the present invention has the advantages of assisting the scorers in reaching a consensus, so as to efficiently achieve the scoring consistency and find out the consistent scoring basis and criteria. In addition, in the present invention, for testees, feature maps can be generated based on the scoring consensus information, and are visualized, and simulated scoring and scoring criteria are provided to the testees by visualized feature maps, so as to assist the testees in finding out learning blind spots and improving implementation defects.

An embodiment of the present invention provides an auxiliary scoring system with scoring consensus, including: a data preprocessing module, an artificial intelligence model module, a scoring consensus computation module, and a computer vision module. The data preprocessing module is configured to provide a plurality of pieces of answer data and a plurality of pieces of scoring data corresponding to the plurality of pieces of answer data as an input dataset. The artificial intelligence model module is configured to input the input dataset to a neural network model to train the neural network model to establish an artificial intelligence model, and generate a plurality of attention maps and a plurality of feature maps by using the artificial intelligence model. The scoring consensus computation module is configured to obtain a piece of scoring consensus information based on the plurality of attention maps and/or the plurality of feature maps. The computer vision module is configured to visualize the scoring consensus information, and mark consensus blocks and non-consensus blocks in the scoring consensus information in different colors.

An embodiment of the present invention provides an auxiliary scoring system with scoring consensus, including: a data digitizing module, a data conversion module, a training module, a validation module, an output module, a scoring consensus computation module, a scoring inferring module, and a computer vision module. The data digitizing module is configured to digitize answer data into digitized answer data. The data conversion module is configured to convert the digitized answer data into vector data and convert scoring data corresponding to the answer data into annotated data, where a plurality of pieces of vector data may compose a vector dataset and a plurality of pieces of annotated data may compose an annotated dataset. The training module is configured to train the neural network model with the vector dataset and the annotated dataset taken as the input dataset. The validation module is configured to validate the neural network model with a five-fold cross-validation, and assess the accuracy of an output prediction of the neural network model to establish an artificial intelligence model. The output module is configured to output a plurality of attention maps and a plurality of feature maps generated by the artificial intelligence model. The scoring consensus computation module is configured to compute scoring consensus information based on the plurality of attention maps. The scoring inferring module is configured to infer the answer data of a testee based on the scoring consensus information by using the artificial intelligence model to generate a plurality of features, and a plurality of scores and a plurality of weights corresponding to the plurality of features, and compute a suggested score based on the plurality of scores and the plurality of weights. The computer vision module is configured to visualize the scoring consensus information, distinguish consensus blocks from non-consensus blocks in the scoring consensus information by different colors, visualize the plurality of features, the plurality of scores, and the plurality of weights, and mark the plurality of features in different colors.

An embodiment of the present invention provides an auxiliary scoring method with scoring consensus, including the following steps: providing a plurality of pieces of answer data and a plurality of pieces of scoring data corresponding to the plurality of answer data as an input dataset; inputting the input dataset into a neural network model to train the neural network model; validating the neural network model, and assessing the accuracy of an output prediction of the neural network model to establish an artificial intelligence model; generating a plurality of attention maps and/or a plurality of feature maps by using the artificial intelligence model; obtaining scoring consensus information based on the plurality of attention maps and/or the plurality of feature maps; and visualizing the scoring consensus information, and marking consensus blocks and non-consensus blocks of the scoring consensus information in different colors.

An embodiment of the present invention provides an auxiliary scoring method with scoring consensus, further including the following steps: feeding back the scoring consensus information to the artificial intelligence model; inferring the answer data of a testee based on the scoring consensus information by using the artificial intelligence model to generate a plurality of features, and a plurality of scores and a plurality of weights corresponding to the plurality of features, and computing a suggested score based on the plurality of scores and the plurality of weights, where the plurality of features correspond to a plurality scoring sub-items of a scoring item; and visualizing the plurality of features, the plurality of scores, and the plurality of weights, and marking the plurality of features in different colors. The answer data of the testee is a tooth model, the scoring item is aesthetics, and the plurality of scoring sub-items include equal gingival margins, line angle smoothness and surface fineness of abutment tooth, margin clarity and continuity, and the amount of grinding on occlusal surfaces.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a block diagram of an auxiliary scoring system with scoring consensus according to an embodiment of the present invention;

FIG. 2 illustrates a schematic diagram of validating a neural network model according to an embodiment of the present invention;

FIG. 3 illustrates a schematic diagram showing attention maps and feature maps generated by an artificial intelligence model according to an embodiment of the present invention;

FIG. 4 illustrates a schematic diagram of computing scoring consensus information by a scoring consensus computation module according to an embodiment of the present invention;

FIG. 5 illustrates a schematic diagram of visualizing scoring consensus information by a computer vision module according to an embodiment of the present invention;

FIG. 6A illustrates a schematic diagram of marking scores of scoring sub-items by a computer vision module according to an embodiment of the present invention;

FIG. 6B illustrates a schematic diagram of marking weights of scoring sub-items by a computer vision module according to an embodiment of the present invention; and

FIG. 7 illustrates a flowchart of an auxiliary scoring method with scoring consensus according to an embodiment of the present invention.

### DETAILED DESCRIPTION

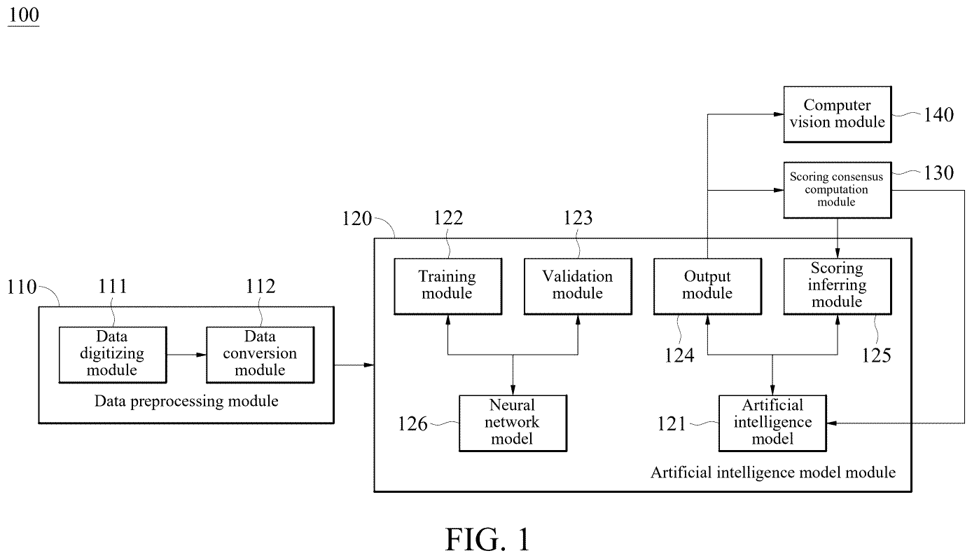

Referring to FIG. 1, FIG. 1 illustrates a block diagram of an auxiliary scoring system 100 with scoring consensus according to an embodiment of the present invention. In the embodiment shown in FIG. 1, the auxiliary scoring system 100 with scoring consensus includes a data preprocessing module 110, an artificial intelligence model module 120, a scoring consensus computation module 130, and a computer vision module 140. The data preprocessing module 110 is configured to provide a plurality of pieces of answer data and a plurality of pieces of scoring data corresponding to the plurality of pieces of answer data as an input dataset to train a neural network model 126. The artificial intelligence model module 120 is configured to receive the input dataset, train the neural network model 126 with the input dataset to establish an artificial intelligence model 121, and generate a plurality of attention maps by using the artificial intelligence model 121. The scoring consensus computation module 130 is configured to compute the plurality of attention maps to obtain a scoring consensus among a plurality of scorers. The computer vision module 140 is configured to visualize the scoring consensus. The auxiliary scoring system 100 with scoring consensus can present the scoring consensus among the plurality of scorers in a visualized manner, so that different scorers can reconcile to a consistent scoring criterion for a subjective scoring item. In addition, the auxiliary scoring system 100 with scoring consensus can provide visualized feature maps to testees, allowing the testees to obtain a weight and score of each scoring sub-item in the subjective scoring item from the visualized feature maps. Therefore, the testees can inspect defects in the answer data by the weights and the scores so as to accurately grasp the improvement direction.

The data preprocessing module 110 includes a data digitizing module 111 and a data conversion module 112. The data preprocessing module 110 may collect a large amount of answer data from the testees and scoring data corresponding to answer data from the scorers to train the neural network model 126. Taking technical examinations or other skill examinations as an example, the answer data may be an article implemented by the testee or skills or performance demonstrated by the testee. The data digitizing module 111 is configured to digitize the answer data into digitized answer data. For example, the answer data is digitized by scanning, photographing, voice recording, and video recording. More specifically, taking dental OSCE as an example, the answer data may be a tooth model implemented by the testee. The tooth model is scanned with an optical scanning device to obtain a 3D digital model of the tooth model. The 3D digital model may be segmented into 2D map faces to obtain a 2D map of the tooth model. The data conversion module 112 is configured to convert the digitized answer data (e.g., the 2D map of the tooth model) into a vector dataset and convert the scoring data corresponding to the answer data into an annotated dataset. In an embodiment, the data conversion module 112 may include a data format converter, for example, a vectorization tool or a label tool. The vectorization tool may convert the digitized answer data (e.g., the 2D map of the tooth model) into vector data. A plurality of pieces of vector data may compose the vector dataset. The label tool may convert the scoring data of the scorer into annotated data. A plurality of pieces of annotated data may compose the annotated dataset. Moreover, the annotated data is also vector data.

The artificial intelligence model module 120 includes an artificial intelligence model 121, a training module 122, a validation module 123, an output module 124, a scoring inferring module 125, and a neural network model 126. The artificial intelligence model module 120 may receive an input dataset. The artificial intelligence model module 120 may train the neural network model 126 with the training module 122. The artificial intelligence model module 120 may validate the neural network model 126 with the validation module 123. The artificial intelligence model module 120 may establish the artificial intelligence model 121 based on the neural network model 126. The artificial intelligence model module 120 may generate attention maps and/or feature maps by using the artificial intelligence model 121, and output the attention maps and/or feature maps to the scoring consensus computation module 130 by the output module 124. The artificial intelligence model module 120 may receive scoring consensus information computed by the scoring consensus computation module 130 or scoring criteria integrated based on the scoring consensus information. The artificial intelligence model module 120 may infer answer data of the testees with the scoring inferring module 125 based on the scoring consensus information by using the artificial intelligence model 121 to obtain a suggested score.

The neural network model 126 may be an architecture consisting of multiple layers of neural networks, for example, a convolutional neural network (CNN), a transformer, U-Net, and Inception. Neural network is a computation model that mimics the structure and function of a biological nervous system, and is also a core technology in the fields of artificial intelligence and machine learning. The convolutional neural network is applicable to image classification, image detection, and image segmentation. Transformer is an attention mechanism-based model that is widely used in natural language processing. U-Net is a convolutional neural network model applicable to image segmentation. Inception is a convolutional neural network model applicable to image classification. In an embodiment, the neural network model 126 may consist of one or more of the following neural network architectures: a convolutional neural network model (CNN), a transformer, U-Net, and Inception.

The neural network model 126 may be used for establishing the artificial intelligence model 121. The neural network model 126 may achieve a target task by optimizing parameters in a training program. The neural network model 126 is iterated and optimized for multiple times to form a final neural network model 126. When an output predicted value generated by the final neural network model 126 meets a standard, that is, the final neural network model 126 may achieve the target task and have stable performance, then the final neural network model 126 may be used for establishing the artificial intelligence model 121.

The training module 122 is configured to train the neural network model 126 with the input dataset. In an embodiment, a vector dataset and an annotated dataset may compose an input dataset. The input dataset may be randomly divided into training datasets, validation datasets, and test datasets, for example: 80% of training datasets and validation datasets, and 20% of test datasets. The above ratio is not fixed and may be adjusted as needed. The training program of the neural network model 126 includes multiple steps. Firstly, the neural network model 126 generates a predicted value by forward propagation, and then computes an error (loss) between the predicted value and a real value by using a loss function. Next, the neural network model 126 adjusts parameters according to gradient information of the loss by backpropagation to gradually reduce the error and improve prediction ability.

The validation module 123 is configured to validate the neural network model 126 with a five-fold cross-validation 201, and assess the similarity between an output prediction of the neural network model 126 and the real value of test data. In an embodiment, the neural network model 126 is validated with the training dataset and the validation dataset in a validation program of five-fold cross-validation 201. Whether the neural network model 126 meets the standard is assessed with the test dataset. The performance of the neural network model 126 on unknown data is assessed with the test dataset to avoid over-fitting or under-fitting. The neural network model 126 is iterated and optimized for multiple times to form a final neural network model 126. An artificial intelligence model 121 is established based on the final neural network model 126.

The output module 124 is configured to transmit a plurality of attention maps and/or a plurality of feature maps generated by the artificial intelligence model 121 to the scoring consensus computation module 130. The artificial intelligence model 121 established by training the neural network model 126 according to a large amount of answer data of the testees and the scoring data corresponding to the answer data from the scorers may generate the attention maps and/or feature maps for each scorer. In an embodiment, the input dataset (e.g., the 2D map of the tooth model) may be understood by a transformer encoder of the artificial intelligence model 121. A deep understanding of the input dataset may be formed after gradual processing by a multi-layer neural network. In this process, by computing attention scores, the model can assess which parts of the input dataset are most critical to the overall understanding, so as to generate a corresponding attention map. By visualizing the attention maps, critical blocks in the data may be intuitively observed and recognized.

The scoring inferring module 125 is configured to receive the scoring consensus of the scoring consensus computation module 130, feed back the scoring consensus to the artificial intelligence model 121, and infer the answer data of the testees based on the scoring consensus information by using the artificial intelligence model 121 to obtain a suggested score. In an embodiment, the answer data of the testees is inferred based on the scoring consensus information by using the artificial intelligence model 121, which may generate a plurality of features, scores of the plurality of features, and weights of the plurality of features. The plurality of features correspond to a plurality of scoring sub-items in a scoring item; the scores of the plurality of features correspond to the scores of the plurality of scoring sub-items; and the weights of the plurality of features correspond to weights of the plurality of scoring sub-items. The suggested score is computed based on the scores of the plurality of scoring sub-items and the weights of the plurality of scoring sub-items. In an embodiment, the formula for computing the suggested score is as follows:

$$Scoring = {\sum }_{i = 0}^{N} {Score}_{i} * {weight}_{i}$$

where Scoring represents the suggested score; Scoreᵢ represents the score of each scoring sub-item; weightᵢ represents the weight of each scoring sub-item; and N represents the number of the scoring sub-items.

The scoring consensus computation module 130 is configured to compute the plurality of attention maps to obtain a scoring consensus. In an embodiment, the scoring consensus computation module 130 includes a union computation unit, an intersection computation unit, and a difference set computation unit. The scoring consensus computation module 130 may receive a plurality of attention maps and/or feature maps transmitted by the output module 124. In an embodiment, the plurality of attention maps and/or feature maps may be superimposed. The scoring consensus information is computed by a union computation method, an intersection computation method, a difference set computation method and the like. The scoring criteria are integrated based on the scoring consensus information. By the scoring criteria, different scorers may reconcile to a consistent scoring criterion for subjective scoring items (e.g., aesthetics or delicacy).

The scoring consensus computation module 130 computes a union of the plurality of attention maps and/or feature maps by using the union computation unit to obtain comprehensive information of a plurality of scorers. The scoring consensus computation module 130 computes an intersection of the union of the plurality of attention maps and/or feature maps and each attention map and/or feature map by using the intersection computation unit to obtain consensus information of each scorer on the comprehensive information. The scoring consensus computation module 130 computes a difference set of the union of the plurality of attention maps and/or feature maps and each attention map and/or feature map by using the difference set computation unit to obtain non-consensus information (or difference information) of each scorer on the comprehensive information. In summary, the scoring consensus computation module 130 may obtain scoring consensus information based on computing the union, intersection, and difference set of the attention maps and/or feature maps. The scoring consensus information includes the comprehensive information, the consensus information, and the non-consensus information.

In an embodiment, the scoring consensus computation module 130 may also feed back the scoring consensus information to the artificial intelligence model 121. In addition, the scoring consensus computation module 130 may also integrate the scoring criteria based on the scoring consensus information, and then feed back the scoring criteria to the artificial intelligence model 121. In an embodiment, the scoring consensus information or the scoring criteria may be converted into a knowledge set to serve as reference data for the inference of the artificial intelligence model 121. By the scoring consensus information or the scoring criteria, different scorers can employ the consistent scoring criteria for the same skill or performance, thereby reducing subjective differences.

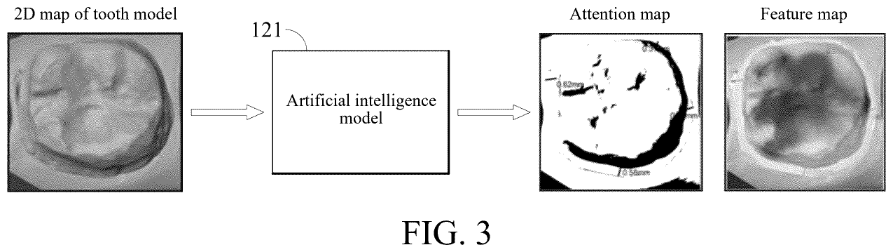

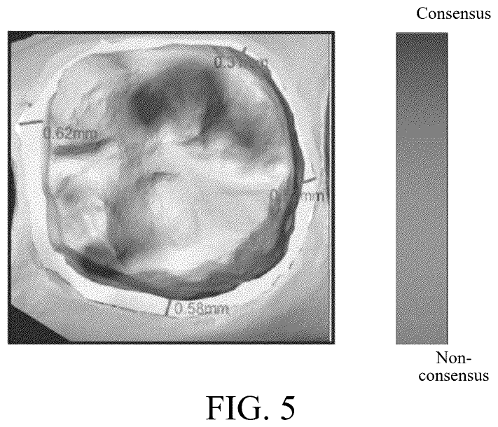

The computer vision module 140 is configured to visualize the scoring consensus of the plurality of scorers. In an embodiment, the computer vision module 140 visualizes the attention map and/or the feature map into a visualized attention map and/or visualized feature map by using a computer vision library. For example, the critical (or important) blocks in the scoring item are marked in white on the visualized attention map (the attention map as shown in FIG. 3). A plurality of features are marked in different colors on the visualized feature map. On the visualized feature map, a plurality of features correspond to a plurality of scoring sub-items of the scoring item. The darker the color, the higher the degree of influence (the feature map as shown in FIG. 3). In an embodiment, the computer vision module 140 marks consensus blocks and non-consensus blocks of the scoring consensus information in different colors on the visualized attention map and/or the visualized feature map. For example, the consensus blocks are marked in red, and the non-consensus blocks are marked in green (as shown in FIG. 5). On the one hand, the visualized presentation of the consensus blocks and non-consensus blocks can help the scorers to quickly find out common scoring criteria or rules in a consensus conference, so as to assist the scorers in reaching a consensus and achieve consistency in scoring. On the other hand, the visualized presentation of the plurality of scoring sub-items, the scores of the plurality of scoring sub-items, and the weights of the plurality of scoring sub-items can provide the testees with simulated scoring and scoring criteria, so as to assist the testees in finding out learning blind spots and improving implementation defects.

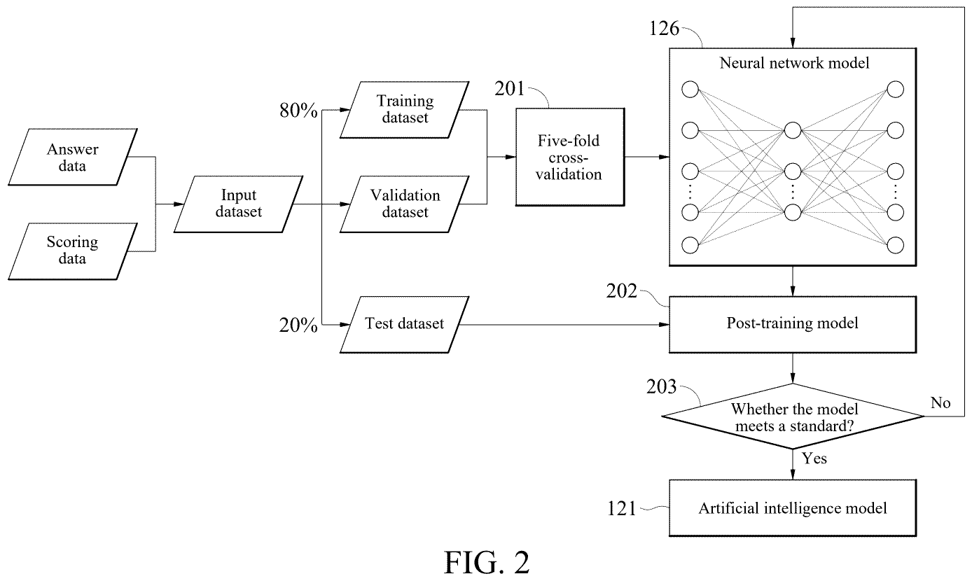

Referring to FIG. 2, FIG. 2 illustrates a schematic diagram of validating a neural network model 126 according to an embodiment of the present invention. In the embodiment shown in FIG. 2, the training and validation process of establishing the artificial intelligence model 121 by the neural network model 126 is illustrated. In an embodiment, the answer data (e.g., digitized files of the tooth model implemented by the testee) and the scoring data (e.g., the scores of an aesthetics item of the tooth model from the scorers) may be inputted as the input dataset to train the neural network model 126, so that the neural network model 126 finally formed has the ability to review the aesthetics of the tooth model. Then, the artificial intelligence model 121 is established by the neural network model 126 finally formed.

In an embodiment, the input dataset may be divided into 80% of training-validation datasets and 20% of test datasets. The training-validation dataset is used for cross-validating the neural network model 126. The test dataset is used for testing the performance of the neural network model 126 finally formed. In the validation program of the five-fold cross-validation 201, the training-validation dataset may be divided into five equivalent subsets. In each iteration, one of the five subsets is selected in turn as a validation dataset, and the remaining four subsets are combined together as the training dataset, thereby forming five different dataset combinations. Each dataset combination is inputted into the neural network model 126 in turn for training and validation, so as to adjust model parameters. After training, the performance of the neural network model 126 is assessed with the test dataset. The neural network model 126 with the best performance in five iterations is selected as a post-training model 202. Alternatively, the post-training model 202 is selected according to an average value of assessment indicators of the neural network model 126.

In an embodiment, the validation program of the five-fold cross-validation 201 further includes a judgment step 203 of whether the model meets the standard. The judgment step is used for judging whether the post-training model 202 (or the neural network model 126 finally formed) meets the standard. The test dataset is inputted into the post-training model 202 for testing, so as to assess the similarity between an output predicted value of the post-training model 202 and the real value of the test data. When the similarity is greater than or equal to a threshold value, the artificial intelligence model 121 may be established based on the post-training model 202. When the similarity is less than the threshold value, the neural network model 126 is further trained. In an embodiment, the similarity may use assessment indicators such as accuracy, mean square error, cross-entropy loss, etc. In an embodiment, the threshold value may be set to 90%. That is, when the similarity between the output predicted value of the post-training model 202 and the real value of the test data is greater than or equal to 90%, it means that the post-training model 202 meets the standard (for example, it has the ability to review the aesthetics item of the tooth model). At this time, the artificial intelligence model 121 may be established based on the post-training model 202.

Referring to FIG. 3, FIG. 3 illustrates a schematic diagram showing attention maps and feature maps generated by an artificial intelligence model 121 according to an embodiment of the present invention. In the embodiment shown in FIG. 3, the artificial intelligence model 121 has the ability to review the aesthetics item of the tooth model. The 2D map of the tooth model is inputted to the artificial intelligence model 121 for analysis and processing. The attention map and feature map for reviewing the aesthetics item of the tooth model are generated by the artificial intelligence model 121. Image block information of the tooth model is analyzed and understood by the transformer encoder in the artificial intelligence model 121. The deep understanding of the aesthetics item of the tooth model is gradually established after processing by the multi-layer neural network. In this process, the artificial intelligence model 121 will compute attention scores to assess which parts are most critical to understanding the aesthetics item in the image block information of the whole tooth model, and generate the attention map based on the attention scores. The visualized attention map may be used for highlighting critical (or important) blocks related to the aesthetics item. In an embodiment, the attention map generated by the artificial intelligence model 121 may be visualized, and the critical (or important) blocks may be distinguished by colors on the visualized attention map, so as to assist the scorers in more accurately understanding the critical (or important) blocks of the aesthetics items of the tooth model. In the attention map as shown in FIG. 3, a white part represents the critical (or important) block of the aesthetics item of the tooth model.

In addition, after the artificial intelligence model 121 analyzes the correlation between the image block information of the tooth model, feature vectors of original image blocks will be gradually updated, and relevant information from other image blocks is fused, thereby forming higher-level and more representative features. Finally, these features are integrated into a feature map. In an embodiment, the feature map generated by the artificial intelligence model 121 may be visualized. A plurality of features extracted by the artificial intelligence model 121 may be distinguished by different colors on the visualized feature map. The plurality of features correspond to the plurality of scoring sub-items for reviewing the aesthetics item of the tooth model. In the feature map as shown in FIG. 3, the plurality of features are marked in different colors to distinguish positions of the plurality of scoring sub-items. The plurality of scoring sub-items include, for example, equal gingival margins, line angle smoothness and surface fineness of abutment tooth, margin clarity and continuity, the establishment of form and tooth gap pattern and the like.

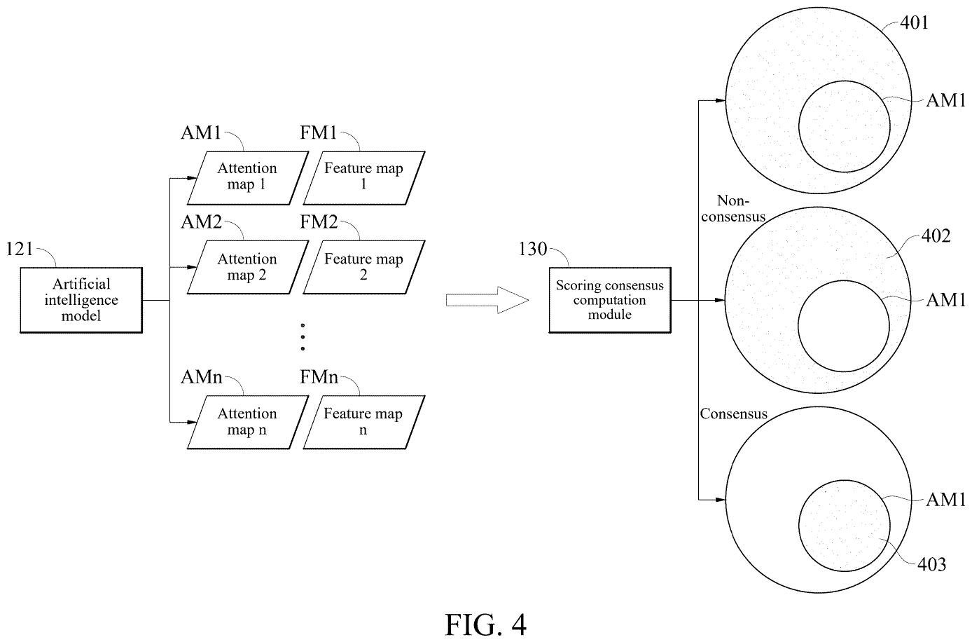

Referring to FIG. 4, FIG. 4 illustrates a schematic diagram of computing scoring consensus information by a scoring consensus computation module 130 according to an embodiment of the present invention. In the embodiment shown in FIG. 4, the artificial intelligence model 121 has the ability to review the aesthetics item of the tooth model after being trained by a large amount of answer data of the testees and the scoring data corresponding to the answer data from the scorers. The artificial intelligence model 121 may generate a plurality of attention maps (e.g., AM1, AM2, . . . , AMn) and a plurality of feature maps (e.g., FM1, FM2, . . . , FMn) for reviewing the aesthetics item of the tooth model. Each attention map and/or feature map correspond(s) to a scorer. For example, the attention map 1 (AM1) and the feature map 1 (FM1) correspond to the scorer 1, the attention map 2 (AM2) and the feature map 2 (FM2) correspond to the scorer 2, and so on. In other words, the artificial intelligence model 121 may generate the attention map and the feature map for each scorer for reviewing the aesthetics item of the tooth model.

In the embodiment as shown in FIG. 4, the scoring consensus computation module 130 has a union computation unit, an intersection computation unit, and a difference set computation unit. The above-mentioned computation units may be implemented by using a processor (e.g., CPU or GPU). In an embodiment, the scoring consensus computation module 130 may firstly superimpose or merge or aggregate the plurality of attention maps and/or feature maps generated by the artificial intelligence model 121, and then integrate the consensus information and the non-consensus information among the scorers with the union computation unit, the intersection computation unit, and the difference set computation unit to obtain scoring consensus information.

In an embodiment, the scoring consensus computation module 130 computes the union of the plurality of attention maps (e.g., AM1, AM2, . . . , AMn) and/or the plurality of feature maps (e.g., FM1, FM2, . . . , FMn) by using the union computation unit to obtain a union result 401. According to the union result 401, the comprehensive information of a plurality of scorers may be obtained. The comprehensive information represents the critical part of the scoring item (e.g., the aesthetics item of the tooth model) that any scorer considers. In an embodiment, the scoring consensus computation module 130 computes a difference set of the union result 401 and the attention map 1 (AM1) and/or the feature map 1 (FM1) by using the difference set computation unit to obtain a difference set result 402. According to the difference set result 402, the non-consensus information of the scorer 1 on the comprehensive information may be obtained. The non-consensus information represents the non-consensus (or difference) in the critical part of the scoring item (e.g., the aesthetics item of the tooth model) that individual scorers consider between the individual scorers and all other scorers. In an embodiment, the scoring consensus computation module 130 computes an intersection of the union result 401 and the attention map 1 (AM1) and/or feature map 1 (FM1) by using the intersection computation unit to obtain an intersection result 403. According to the intersection result 403, the consensus information of the scorer 1 on the comprehensive information may be obtained. The consensus information represents the consensus in the critical part of the scoring item (e.g., the aesthetics item of the tooth model) that individual scorers considers between the individual scorers and any other scorer. In summary, the scoring consensus computation module 130 obtains scoring consensus information based on the computation of the union, intersection, and difference set of the attention maps and/or feature maps. The scoring consensus information includes the comprehensive information, the consensus information, and the non-consensus information.

Referring to FIG. 5, FIG. 5 illustrates a schematic diagram of visualizing scoring consensus information by a computer vision module 140 according to an embodiment of the present invention. In the embodiment shown in FIG. 5, the scoring consensus computation module 130 may transmit the scoring consensus information, including the comprehensive information, the consensus information, and the non-consensus information, obtained by computing the attention maps and/or feature maps to the computer vision module 140, and visualize the scoring consensus information obtained by computing the attention maps and/or feature maps by the computer vision module 140. In an embodiment, the computer vision module 140 visualizes, by using a computer vision library, the scoring consensus information obtained by computing the attention maps and/or feature maps, and mark consensus blocks and non-consensus blocks in different colors on the scoring consensus information of the attention maps and/or feature maps. For example, the consensus blocks are marked in red, the non-consensus blocks are marked in green, and the degree of consensus and the degree of non-consensus are presented in shades of color. In an embodiment, the comprehensive information based on the attention maps and/or feature maps is visualized, which may present the critical part of the scoring item (e.g., the aesthetics item of the tooth model) that every scorer considers. In an embodiment, the consensus information based on the attention maps and/or feature maps is visualized, which may present the degree of consensus between individual scorers and any scorer on the scoring item (e.g., the aesthetics item of the tooth model). In an embodiment, the non-consensus information based on the attention maps and/or feature maps is visualized, which may present the degree of non-consensus between individual scorers and all the scorers on the scoring item (e.g., the aesthetics item of the tooth model). Therefore, the scorers may inspect their own scoring criteria, as well as their own consensus and non-consensus with other scorers on the scoring criteria. In addition, the visualized scoring consensus information may assist in accelerating the establishment of the scoring consensus among the scorers in the consensus conference, or finding out the consistent scoring basis and criteria to ensure the fairness of the examination.

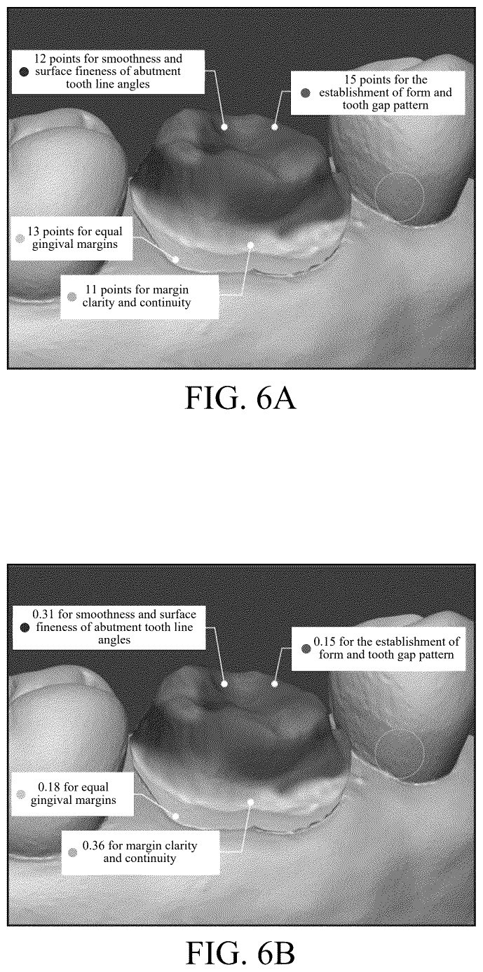

Referring to FIG. 6A and FIG. 6B, FIG. 6A illustrates a schematic diagram of marking scores of scoring sub-items by a computer vision module 140 according to an embodiment of the present invention, and FIG. 6B illustrates a schematic diagram of marking weights for scoring sub-items by a computer vision module 140 according to an embodiment of the present invention. The scoring inferring module infers the answer data of the testees based on the scoring consensus information by using the artificial intelligence model 121 to generate a plurality of features of a scoring item (e.g., the aesthetics item of the tooth model), scores of the plurality of features, and weights of the plurality of features. The plurality of features correspond to a plurality of scoring sub-items (e.g., equal gingival margins, smoothness and surface fineness of abutment tooth line angles, margin clarity and continuity, and the establishment of form and tooth gap pattern) in the scoring item (e.g., the aesthetics item of the tooth model). The scores of the plurality of features correspond to the scores of the plurality of scoring sub-items (for example, 13 points for equal gingival margins, 12 points for line angle smoothness and surface fineness of abutment tooth, 11 points for margin clarity and continuity, and 15 points for the establishment of form and tooth gap pattern). The weights of the plurality of features correspond to the weights of the plurality of scoring sub-items (for example, 0.18 for equal gingival margins, 0.31 for line angle smoothness and surface fineness of abutment tooth, 0.36 for margin clarity and continuity, and 0.15 for the establishment of form and tooth gap pattern).

In an embodiment, the artificial intelligence model 121 may generate the feature maps based on the scoring consensus information and visualize, by the computer vision module 140, the feature maps generated based on the scoring consensus information. The computer vision module 140 may mark the plurality of scoring sub-items corresponding to the plurality of features in different colors on the visualized feature map. In addition, the computer vision module 140 may mark the scores of the plurality of scoring sub-items (as shown in FIG. 6A) and the weights of the plurality of scoring sub-items (as shown in FIG. 6B) on the visualized feature map. In an embodiment, the artificial intelligence model 121 may be trained by using the scoring consensus information, or the scoring consensus information is used as an additional knowledge base to assist the artificial intelligence model 121 in performing inference. Moreover, the feature map generated by the artificial intelligence model 121 based on the scoring consensus information may provide the testees with simulated scoring and scoring criteria in the scoring item (e.g., the aesthetics item of the tooth model), so as to assist the testees in finding out learning blind spots and improving implementation defects.

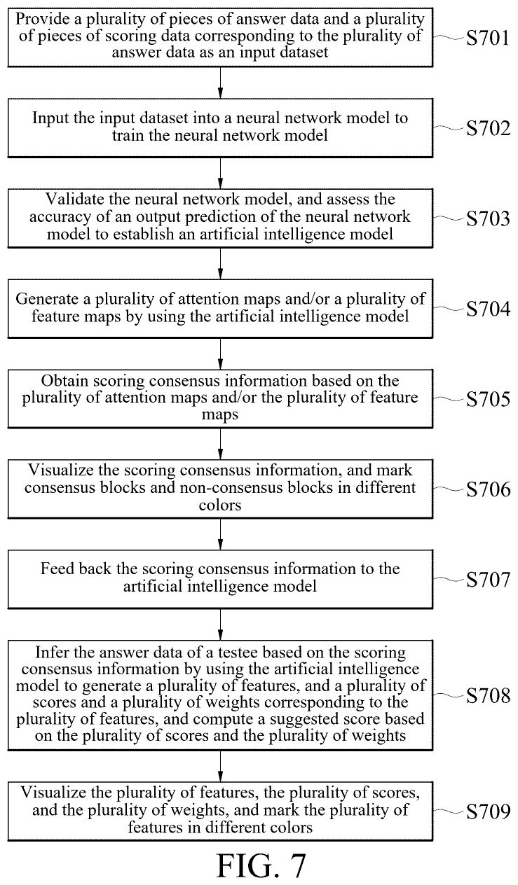

Referring to FIG. 7, FIG. 7 illustrates a flowchart of an auxiliary scoring method with scoring consensus according to an embodiment of the present invention. In the embodiment shown in FIG. 7, the auxiliary scoring method with scoring consensus includes the following steps: providing a plurality of pieces of answer data and a plurality of pieces of scoring data corresponding to the plurality of answer data as an input dataset (step S701); inputting the input dataset into a neural network model 126 to train the neural network model 126 (step S702); validating the neural network model 126, and assessing the accuracy of an output prediction of the neural network model 126 to establish an artificial intelligence model 121 (step S703); generating a plurality of attention maps and/or a plurality of feature maps by using the artificial intelligence model 121 (step S704); obtaining scoring consensus information based on the plurality of attention maps and/or the plurality of feature maps (step S705); visualizing the scoring consensus information, and marking consensus blocks and non-consensus blocks in different colors (step S706); feeding back the scoring consensus information to the artificial intelligence model 121 (step S707); inferring the answer data of a testee based on the scoring consensus information by using the artificial intelligence model 121 to generate a plurality of features, and a plurality of scores and a plurality of weights corresponding to the plurality of features, and computing a suggested score based on the plurality of scores and the plurality of weights (step S708); and visualizing the plurality of features, the plurality of scores, and the plurality of weights, and marking the plurality of features in different colors (step S709).

Although the present invention has been disclosed as above by embodiments, the embodiments are not intended to limit the present invention. Any person skilled in the art can make some changes and embellishments without departing from the spirit and scope of the present invention. Therefore, the scope of protection of the present invention shall be subject to the scope defined in the appended claims.

What is claimed is:

1. An auxiliary scoring system with scoring consensus, comprising:
a data preprocessing module, configured to provide a plurality of pieces of answer data and a plurality of pieces of scoring data corresponding to the plurality of pieces of answer data as an input dataset;
an artificial intelligence model module, configured to input the input dataset to a neural network model to train the neural network model to establish an artificial intelligence model, and generate a plurality of attention maps and a plurality of feature maps by using the artificial intelligence model;
a scoring consensus computation module, configured to obtain a piece of scoring consensus information based on the plurality of attention maps and/or the plurality of feature maps; and
a computer vision module, configured to visualize the scoring consensus information.

2. The auxiliary scoring system with scoring consensus according to claim 1, wherein the data preprocessing module comprises:
a data digitizing module, configured to digitize the answer data into a piece of digitized answer data; and
a data conversion module, configured to convert the digitized answer data into a piece of vector data and convert the scoring data into a piece of annotated data, wherein a plurality of pieces of vector data can compose a vector dataset, and a plurality of pieces of annotated data can compose an annotated dataset.

3. The auxiliary scoring system with scoring consensus according to claim 1, wherein the artificial intelligence model module comprises:
a training module, configured to train the neural network model with a vector dataset and an annotated dataset taken as the input dataset;
a validation module, configured to validate the neural network model with a five-fold cross-validation, and assess the accuracy of an output prediction of the neural network model with a test dataset to establish the artificial intelligence model; and
an output module, configured to transmit the plurality of attention maps and the plurality of feature maps generated by the artificial intelligence model to the scoring consensus computation module, wherein the attention map is generated by understanding the input dataset by a transformer encoder of the artificial intelligence model.

4. The auxiliary scoring system with scoring consensus according to claim 1, wherein the scoring consensus computation module comprises:
a union computation unit, configured to compute a union of the plurality of attention maps to obtain a piece of comprehensive information of a plurality of scorers;
an intersection computation unit, configured to compute an intersection of the union of the plurality of attention maps and each of the attention maps to obtain a piece of consensus information of each of the scorers on the comprehensive information; and
a difference set computation unit, configured to compute a difference set of the union of the plurality of attention maps and each of the attention maps to obtain a piece of non-consensus information of each of the scorers on the comprehensive information,
wherein the scoring consensus information comprises the comprehensive information, the consensus information, and the non-consensus information.

5. The auxiliary scoring system with scoring consensus according to claim 1, wherein the scoring consensus computation module comprises:
a union computation unit, configured to compute a union of the plurality of feature maps to obtain a piece of comprehensive information of a plurality of scorers;
an intersection computation unit, configured to compute an intersection of the union of the plurality of feature maps and each of the feature maps to obtain a piece of consensus information of each of the scorers on the comprehensive information; and
a difference set computation unit, configured to compute a difference set of the union of the plurality of feature maps and each of the feature maps to obtain a piece of non-consensus information of each of the scorers on the comprehensive information,
wherein the scoring consensus information comprises the comprehensive information, the consensus information, and the non-consensus information.

6. The auxiliary scoring system with scoring consensus according to claim 1, wherein the computer vision module visualizes the scoring consensus information by using a computer vision library, and distinguish consensus blocks from non-consensus blocks by different colors.

7. The auxiliary scoring system with scoring consensus according to claim 1, wherein the artificial intelligence model module comprises a scoring inferring module, the scoring inferring module being configured to infer the answer data of a testee based on the scoring consensus information by using the artificial intelligence model to generate a plurality of features, a plurality of scores corresponding to the plurality of features, and a plurality of weights corresponding to the plurality of features, and compute a suggested score based on the plurality of scores and the plurality of weights, the plurality of features corresponding to a plurality of scoring sub-items of a scoring item.

8. The auxiliary scoring system with scoring consensus according to claim 7, wherein the computer vision module is configured to visualize the plurality of features, the plurality of scores corresponding to the plurality of features, and the plurality of weights corresponding to the plurality of features, and distinguish the plurality of features by different colors.

9. The auxiliary scoring system with scoring consensus according to claim 8, wherein the answer data of the testee is a tooth model, the scoring item is aesthetics, and the plurality of scoring sub-items comprise equal gingival margins, line angle smoothness and surface fineness of abutment tooth, margin clarity and continuity, and the amount of grinding on occlusal surfaces.

10. An auxiliary scoring system with scoring consensus, comprising:
a data digitizing module, configured to digitize a piece of answer data into a piece of digitized answer data;
a data conversion module, configured to convert the digitized answer data into a piece of vector data and convert a piece of scoring data corresponding to the answer data into a piece of annotated data, wherein a plurality of pieces of vector data can compose a vector dataset and a plurality of pieces of annotated data can compose an annotated dataset;
a training module, configured to train a neural network model with the vector dataset and the annotated dataset taken as an input dataset;
a validation module, configured to validate the neural network model with a five-fold cross-validation, and assess the accuracy of an output prediction of the neural network model to establish an artificial intelligence model;
an output module, configured to output a plurality of attention maps and a plurality of feature maps generated by the artificial intelligence model;
a scoring consensus computation module, configured to compute a piece of scoring consensus information based on the plurality of attention maps;
a scoring inferring module, configured to infer the answer data of a testee based on the scoring consensus information by using the artificial intelligence model to generate a plurality of features, and a plurality of scores and a plurality of weights corresponding to the plurality of features, and compute a suggested score based on the plurality of scores and the plurality of weights; and
a computer vision module, configured to visualize the scoring consensus information, distinguish consensus blocks from non-consensus blocks by different colors, visualize the plurality of features, the plurality of scores and, the plurality of weights, and mark the plurality of features in different colors.

11. An auxiliary scoring method with scoring consensus, comprising:
(a) providing a plurality of pieces of answer data and a plurality of pieces of scoring data corresponding to the plurality of pieces of answer data as an input dataset;
(b) inputting the input dataset into a neural network model to train the neural network model;
(c) validating the neural network model, and assessing the accuracy of an output prediction of the neural network model to establish an artificial intelligence model;
(d) generating a plurality of attention maps and a plurality of feature maps by using the artificial intelligence model;
(e) obtaining a piece of scoring consensus information based on the plurality of attention maps and/or the plurality of feature maps; and
(f) visualizing the scoring consensus information, and marking consensus blocks and non-consensus blocks in different colors.

12. The auxiliary scoring method with scoring consensus according to claim 11, wherein the step (a) comprises:
digitizing the answer data into a piece of digitized answer data; and
converting the digitized answer data into a piece of vector data and converting the scoring data corresponding to the answer data into a piece of annotated data, wherein a plurality of pieces of vector data can compose a vector dataset and a plurality of pieces of annotated data can compose an annotated dataset.

13. The auxiliary scoring method with scoring consensus according to claim 12, wherein the step (b) comprises:
training the artificial intelligence model with the vector dataset and the annotated dataset taken as the input dataset.

14. The auxiliary scoring method with scoring consensus according to claim 11, wherein the step (c) comprises:
validating the artificial intelligence model with a five-fold cross-validation; and
assessing the artificial intelligence model with a test dataset.

15. The auxiliary scoring method with scoring consensus according to claim 11, wherein the step (d) comprises:
understanding the input dataset by using a transformer encoder to generate the plurality of attention maps.

16. The auxiliary scoring method with scoring consensus according to claim 11, wherein the step (e) comprises:
computing a union of the plurality of attention maps to obtain a piece of comprehensive information of a plurality of scorers;
computing an intersection of the union of the plurality of attention maps and each of the attention maps to obtain a piece of consensus information of each of the scorers on the comprehensive information; and
computing a difference set of the union of the plurality of attention maps and each of the attention maps to obtain a piece of non-consensus information of each of the scorers on the comprehensive information,
wherein the scoring consensus information comprises the comprehensive information, the consensus information, and the non-consensus information.

17. The auxiliary scoring method with scoring consensus according to claim 11, wherein the step (e) comprises:
computing a union of the plurality of feature maps to obtain a piece of comprehensive information of a plurality of scorers;
computing an intersection of the union of the plurality of feature maps and each of the feature maps to obtain a piece of consensus information of each of the scorers on the comprehensive information; and
computing a difference set of the union of the plurality of feature maps and each of the feature maps to obtain a piece of non-consensus information of each of the scorers on the comprehensive information,
wherein the scoring consensus information comprises the comprehensive information, the consensus information, and the non-consensus information.

18. The auxiliary scoring method with scoring consensus according to claim 11, wherein the step (f) comprises:
visualizing the scoring consensus information by using a computer vision library, and distinguish consensus blocks from non-consensus blocks by different colors.

19. The auxiliary scoring method with scoring consensus according to claim 11, further comprising:
(g) feeding back the scoring consensus information to the artificial intelligence model;
(h) inferring the answer data of a testee based on the scoring consensus information by using the artificial intelligence model to generate a plurality of features, and a plurality of scores and a plurality of weights corresponding to the plurality of features, and computing a suggested score based on the plurality of scores and the plurality of weights, wherein the plurality of features correspond to a plurality scoring sub-items of a scoring item; and
(i) visualizing the plurality of features, the plurality of scores, and the plurality of weights, and marking the plurality of features in different colors.

20. The auxiliary scoring method with scoring consensus according to claim 19, wherein the answer data of the testee is a tooth model, the scoring item is aesthetics, and the plurality of scoring sub-items comprise equal gingival margins, line angle smoothness and surface fineness of abutment tooth, margin clarity and continuity, and the amount of grinding on occlusal surfaces.
